(19) United States
(12) Patent Application Publication
(10) Pub. No.: US 2002/0192831 A1
(43) Pub. Date: Dec. 19, 2002

(54) Method of controlling the hydrocarbon content of a vapor circulating in an installation fitted with a vapor intake system

(76) Inventor: Jacques Fournier, Bretigny Sur Orge (FR)

Correspondence Address: RANDALL J. KNUTH P.C., 3510-A STELLHORN ROAD, FORT WAYNE, IN, 46815-4631, US

(21) Appl. No.: 10/060,538

(22) Filed: Jan. 30, 2002

(30) Foreign Application Priority Data
Apr. 6, 2001 (FR) 01 04 704

(51) Int. Cl. G05B 21/00; G01N 33/00; G01N 1/22

(52) U.S. Cl. 436/139; 700/274; 436/55; 436/143; 436/148; 436/181; 700/266; 73/23.2; 73/30.01; 73/30.02; 73/30.03

(57) ABSTRACT

A method of controlling the hydrocarbon content of a possible explosive mixture of air/hydrocarbon vapor circulating from an intake point into an installation fitted with a vapor intake system comprising a vapor intake circuit incorporating a suction pump enabling vapor to be circulated at a vapor flow rate Q_{V}. A device is connected to the vapor intake circuit to determine the hydrocarbon content of the aspirated vapor comprising a combination of a flow meter on the one hand and a sensor for measuring relative pressure by reference to atmospheric pressure P_{A} on the other. The hydrocarbon content of the vapor circulating in the vapor intake circuit is determined by taking account of the density and the viscosity of this vapor, which is derived on the basis of a characteristic linked to the loss in air pressure previously stored in memory.

### BACKGROUND OF THE INVENTION

1. Field of the Invention

The present invention relates to a method of controlling the hydrocarbon content of a mixture of air/hydrocarbon vapor circulating from an intake point into an installation fitted with a vapor intake system.

The specific purpose of this method is to rule out any risk of explosion following the intake of an explosive mixture consisting of air with a hydrocarbon content of between 2% and 8%.

2. Description of the Related Art

As stated above, an installation of this type is susceptible to risks of explosion if an explosive mixture of air containing 2 to 8% of hydrocarbons is vacuumed in.

Various manufacturers have attempted to remedy these disadvantages by measuring a characteristic of the aspirated mixture at each instant but nobody to date has proposed a system that is entirely satisfactory for this purpose.

For example, patent specification EP-0 985 634 proposed using optical fibre sensors specifically for analyzing vapors; the reliability of these optical sensors is open to question, however, since the aspirated vapors are often laden with dust which can be deposited on the sensors and distort the measurements.

Patent document U.S. Pat. No. 5,944,067 proposed detecting the hydrocarbon content in the aspirated air by using heat-conductive sensors.

However, sensors of this type generally have too long a response time.

Patent document FR-2 790 255 proposed measuring the hydrocarbon content in the aspirated air by means of density sensors using a process based on determining the velocity of sound in the vapors, which has the disadvantage of being very complex.

Patent specification U.S. Pat. No. 5,860,457 proposed measuring the density of aspirated vapors using two flow meters, namely a density flow meter and a venturi fitted with a differential pressure sensor. This latter sensor is particularly complex given the low pressure differential measured; furthermore, the fact of using two flow meters in parallel makes the task of ascertaining real flow rates and hence density more complicated.

Patent document U.S. Pat. No. 5,038,838 proposed calculating the absolute density of aspirated vapor using an empirical formula and to do so by measuring a pressure correlated to a specific hydraulic resistance on a level with the dispensing gun and working on the assumption that the density of the fluid (or its velocity) is determined by the rotation speed of the pump vacuuming in the vapors, which is a variable speed pump.

A method of this type may work in theory but not in practice, given that all pumps have an internal leakage which varies with flow rate, which means that the result will necessarily be flawed.

The present invention enables the disadvantages outlined above to be remedied by proposing a method of monitoring the hydrocarbon content of vapor circulating in the system for recovering vapor emitted in a fuel dispensing installation that is perfectly reliable, inexpensive in terms of cost price and has a short response time while at the same time not being susceptible to problems caused by dirt or dust entrained with the aspirated vapor.

### SUMMARY OF THE INVENTION

An installation of the present invention type comprises at least in one form,

a vapor intake circuit comprising a suction pump enabling the vapor to circulate at a vapor flow rate Q_{V} and

an electronic-control system provided with a microprocessor co-operating with means for regulating the vapor flow rate Q_{V}, in particular with a proportional solenoid valve connected into the vapor intake circuit.

In accordance with the invention, this method is essentially characterized by the fact that a device is connected into the vapor intake circuit in order to determine the hydrocarbon content of the aspirated vapor, which consists of a combination of firstly, a flow meter and secondly, a sensor which measures the relative pressure, in particular by reference to the atmospheric pressure P_{A}.

This flow meter and this pressure sensor are robust and inexpensive devices.

For the purposes of the invention, the device for determining the hydrocarbon content of the aspirated vapor is connected to the electronic control system so that it can generate instantaneous values for the vapor flow rate Q_{VLU} indicated by the flow meter on the one hand and the relative pressure δP on the other, indicated by the pressure sensor and representing the loss in pressure in the part of the vapor intake circuit disposed between the intake point on the one hand and the pressure sensor and flow meter on the other.

The installation is calibrated with air beforehand in order to determine a characteristic linked to the loss in air pressure in the part of the vapor intake circuit disposed between the intake point on the one hand and the pressure sensor and flow meter on the other and this characteristic is stored in memory.

During normal operation, the values of the vapor flow rate Q_{VLU} and the relative pressure δP are measured at regular intervals. Using the vapor flow rate QVLU as a basis, the actual instantaneous flow rate is calculated and the pressure effect is corrected by the formula:

$${Q}_{v} = {Q}_{VLU} * ( \frac{δ P}{{P}_{A}} + 1 )$$

The hydrocarbon content of the vapor circulating in the vapor intake circuit is determined by taking account of the density ρ and the viscosity μ of this vapor, which are derived from the characteristic linked to the loss in air pressure stored in memory beforehand and a command or an alarm is triggered or the installation is shut down if this hydrocarbon content is found to be within a predetermined range, in particular within a range presenting a risk of explosion.

By virtue of a first embodiment of the invention, the characteristic linked to the drop in air pressure in the part of the vapor intake circuit disposed between the intake point on the one hand and the pressure sensor and flow meter on the other is the resistance R defined by the equation:

$$R = \frac{δ P}{{Q}_{V}^{x}}$$

in which

δP represents the loss in pressure expressed in Pascals,

Q_{V} represents the vapor flow rate expressed in m³/s and

x represents a parameter equal to 7/4 in theory and approximately 1.8 in practice.

Furthermore, it is known that in a passage with a length that is very much greater than the diameter, which is the case in this particular instance, the drop in pressure δP is also defined by the equation:

$$δ P = C [ \frac{L * {ρ}^{3 / 4} * {Q}_{V}^{x} * {μ}^{1 / 4}}{{d}^{19 / 4}} ]$$

in which:

L represents the length of the part of the circuit in question expressed in meters,

d represents the diameter in question, being a constant of this part of the circuit, expressed in meters,

μ represents the viscosity of the vapor expressed in Pa.s,

ρ represents the density of the vapor expressed in g/l and

C represents a parameter equal to 0.2414.

These two equations prove that the resistance R depends only on the geometry of the installation and the nature of the vapor circulating in it, but not on the vapor flow rate.

Consequently, the hydrocarbon content of the aspirated air can be determined by comparing the resistance values R during the prior calibration step with air on the one hand and during normal operation on the other.

To this end and by virtue of another essential feature of this first embodiment of the invention:

A table T[Q_{V}, Q_{V}^{x}] is computed in which a value Q_{V}^{x} is correlated with different vapor flow rates Q_{V} between 0 and Q_{VMAX} and this table is stored in memory, and during the prior step of calibrating the installation with air, the suction pump is activated and the regulating means are controlled in order to obtain several different vapor flow rates Q_{V}.

The relative pressure δP corresponding to these vapor flow rates Q_{V} is measured and a value for the air resistance R in the part of the vapor intake circuit disposed between the intake point on the one hand and the pressure sensor and flow meter on the other is derived for each one from the table T[Q_{V}, Q_{V}^{x}].

The average R0 of the different values R thus obtained is calculated and stored in memory, and during normal operation, the values of the vapor flow rate Q_{VLU} and the relative pressure δP are measured at regular intervals, in particular every ½ second.

The real vapor flow rate Q_{V} is calculated from the vapor flow rate QVLU using the formula:

$${Q}_{V} = {Q}_{VLU} ( \frac{δ P}{{P}_{A}} + 1 )$$

the value Q_{V}^{x} is derived from the table T [Q_{V}, Q_{V}^{x}],

the value of the vapor resistance R1 in the part of the intake circuit disposed between the intake point on the one hand and the pressure sensor and flow meter on the other is calculated and

the vapor resistance R1 is compared with the air resistance R0.

It should be pointed out that the accuracy of the result obtained is dependent on the number of values Q_{V}^{x} calculated between 0 and Q_{VMAX}, which defines the intervals of the table T[Q_{V}, Q_{V}^{x}]. In accordance with the invention, a command or an alarm is triggered or the in stallation is shut down if the ratio R1/R0 is found to be within a predetermined range, in particular if it is found that:

R1≦kR0

The parameter k is a parameter which allows the upper limit 5 of explosiveness corresponding to a vapor Vₑₓₚ with an 8% hydrocarbon content to be taken into account.

In view of the aforementioned equations, this parameter k is equal to:

$$k = {( \frac{{P}_{V exp}}{{P}_{air}} )}^{3 / 4} {( \frac{{μ}_{V exp}}{{μ}_{air}} )}^{1 / 4} \approx 1.063$$

By, virtue of a second embodiment of the invention, which has an advantage in that it does not require the air resistance and the vapor resistance to be calculated in the part of the intake circuit disposed between the intake point on the one hand and the pressure sensor and flow meter on the other, the method comprises the following sequence of steps:

during the prior step of calibrating the installation with air, the suction pump is activated and the regulating means are activated step by step so as to vary the air flow circulating in the vapor intake circuit,

with each step, the values of the vapor flow rate Q_{VLU} and the relative pressure δP are measured,

the vapor flow rate Q_{V} is calculated from the vapor flow rate Q_{VLU} using the formula:

$${Q}_{V} = {Q}_{VLU} ( \frac{δ P}{P a} + 1 )$$

a table T0[δP, Qᵥ] is established, representing the characteristic linked to the drop in air pressure in the part of the vapor intake circuit disposed between the intake point on the one hand and the pressure sensor and flow meter on the other and this table T0[δP, Qᵥ] is stored in memory,

during normal operation, the values of the vapor flow rate Q_{VLU} and relative pressure δP are measured at regular intervals, for example every ½ second,

the real vapor flow rate Q_{V} is calculated from the vapor flow rate Q_{VLU} by the formula:

$${Q}_{V} = {Q}_{VLU} ( \frac{δ P}{P a} + 1 )$$

for each vapor flow rate Qᵥ, the table T0[δP, Qᵥ] is searched for a relative pressure δPₐᵢᵣ corresponding to the same rate of air flow,

the relative pressures δP and δPₐᵢᵣ are compared by calculating a factor λ defined by the equation:

$$λ = \frac{δ P - δ {P}_{air}}{δ {P}_{air}}$$

As stated above, the relative pressure δP corresponding to the drop in pressure in the part of the vapor intake circuit disposed between the intake point on the one hand and the pressure sensor and flow meter on the other is also defined by the equation:

$$δ P = C [ \frac{L * {ρ}^{3 / 4} * {Q}_{V}^{x} * {μ}^{1 / 4}}{{d}^{19 / 4}} ]$$

in which, if δP is expressed in Pascal,

L represents the length of the part of the circuit in question expressed in m,

d represents the diameter in question, being a constant of this part of the circuit, expressed in m,

μ represents the viscosity of the vapor expressed in Pa.s,

ρ represents the density of the vapor expressed in g/l,

C represents a parameter equal to 0.2414,

Q_{V} represents the vapor flow rate expressed in m³/s and

x represents a parameter equal to 7/4 in theory and approximately 1.8 in practice.

The factor λ is then also defined by the equation:

$$λ = \frac{{( {ρ}^{3 / 4} * {μ}^{1 / 4} )}_{vapor}}{{( {ρ}^{3 / 4} * {μ}^{1 / 4} )}_{air}} - 1$$

Consequently, given that the values of ρₐᵢᵣ and μₐᵢᵣ are known [ρₐᵢᵣ=1.29 g/l and μₐᵢᵣ=180 micropoises (micropoise=10⁻⁷ Pa.s)] as are the corresponding values in the case of a mixture Vₑₓₚ constituting air with 8% hydrocarbons which corresponds to the upper limit of explosiveness, it may be ascertained that λₑₓₚ≈0.063.

Accordingly, in this second embodiment of the invention, a command or alarm is triggered or the installation is shut down if λ is found to be within a predetermined range, in particular if it is found that:

λ≦λₑₓₚ≈0.063

With these two embodiments of the invention, it is of particular advantage to run a regular automatic calibration of the installation with air in order to update the characteristic linked to the drop in air pressure in the part of the vapor intake circuit disposed between the intake point on the one hand and the pressure sensor and flow meter on the other. Accordingly, allowance can be made for any modifications in the installation (ageing and wear of the pumps, gradual incrustation of the pipework, etc.).

By virtue of another feature of the invention, the effects of temperature are corrected.

By virtue of yet another feature of the invention, automatic calibrations with air are run at a sufficient frequency to correct the temperature and the associated sensor.

### BRIEF DESCRIPTION OF THE DRAWINGS

The above-mentioned and other features and advantages of this invention, and the manner of attaining them, will become more apparent and the invention will be better understood by reference to the following description of an embodiment of the invention taken in conjunction with the accompanying drawings, wherein:

FIG. 1 is a diagrammatic view of a fuel dispenser utilized with the present invention;

FIG. 2 is a is a diagrammatic view of the fuel dispenser with the present invention illustrated;

FIG. 3 is a an enlarged sectional view of FIG. 2; and

FIG. 4 is a is a diagrammatic view of an alternate embodiment of the present invention.

Corresponding reference characters indicate corresponding parts throughout the several views. The exemplification set out herein illustrates one preferred embodiment of the invention, in one form, and such exemplification is not to be construed as limiting the scope of the invention in any manner.

### DETAILED DESCRIPTION OF THE INVENTION

As a result of a preferred feature of the invention, the installation is an installation for dispensing fuel fitted with a system for recovering any emitted vapor, corresponding to the vapor intake system.

As a standard, an installation of this type generally comprises

a storage tank for the fuel to be dispensed,

a liquid dispensing circuit comprising a distribution pump enabling the fuel to be circulated at a liquid flow rate QL between the storage tank and the fuel tank of a vehicle,

a vapor recovery circuit corresponding to the vapor intake circuit, comprising a recovery pump corresponding to the suction pump enabling any vapor emitted whilst filling the fuel tank to be recovered between the latter and the storage tank at a vapor flow rate Q_{V}.

counting means connected to the liquid dispensing circuit and comprising a liquid meter connected to a pulse generator or encoder enabling a computer to establish the volume and price of the fuel dispensed, which appears in plain text on a display,

a dispensing gun connected to the liquid dispensing circuit and to the vapor recovery circuit and fitted with an end-piece enabling the fuel to be dispensed into the fuel tank of a vehicle and having an annular orifice which allows vapors to be sucked back towards the storage tank and

an electronic control system equipped with a microprocessor, connected to the counting means in order to generate the instantaneous value of the liquid flow rate Q_{L} and co-operating with regulating means connected to the vapor recovery circuit in order to maintain the vapor flow rate Qᵥ at approximately the same rate as the liquid flow rate Q_{L}.

In an installation of this type, the regulating means may be provided in the form of a proportional solenoid valve or alternatively a variable speed pump.

It is a known fact that in certain particular instances, especially if the user does not insert the dispensing gun into the fuel tank correctly, the vapor vacuumed into the vapor recovery circuit may incorporate air, which can cause an explosive mixture to occur.

Furthermore, for several years, automobile manufacturers have been fitting some of their vehicles with systems for processing vapors internally by filtering on activated carbon and when a vehicle fitted with this feature arrives at a fuel dispensing pump with a vapor recovery system, there is generally a risk of pumping vapor with a dangerous concentration of hydrocarbons.

An example of a fuel dispensing installation of the type covered by the invention is illustrated in FIG. 1.

In this drawing, the installation is equipped with a gun 10 enabling liquid fuel to be dispensed via an end-piece 11 and any vapor that is emitted to be sucked in through an annular orifice 12.

The fuel is stored in an underground tank 20 and aspirated by a suction/delivery pump 30 mounted in a liquid dispensing circuit having a distribution line 31 immersed in the tank 20.

At the opposite end of this line 31 from the tank 20, a liquid-vapor separator 35 is provided, downstream of which the fuel flow is channeled into the external part of a coaxial flex-pipe 36 and then dispensed by means of the dispensing gun 10 at a liquid flow rate Q_{L}.

The quantity dispensed is determined by counting means connected into the line 31 which has a meter 40 connected to an encoder 41, a computer 42 and a display 43 indicating the volume and price of the fuel dispensed.

During the dispensing process, a pump 50 mounted on a line 51 allows vapor in the fuel tank during filling to be aspirated through the annular orifice 12 of the dispensing gun into a circuit for recovering emitted vapor; this vapor is then channeled through the central part of the coaxial flex-pipe 36 as far as the liquid/vapor separator 35 and then into the vapor recovery line 51 linking the separator 35 to the storage tank 20.

Consequently, the pump 50 delivers the aspirated vapor back to the tank 20 occupying the exact volume freed by the dispensed fuel so that the pressure in the storage tank 20 remains close to atmospheric pressure P_{A}.

To ensure that emitted vapor is recovered with an efficiency close to 100%, the liquid flow rate Q_{L} must be the same as the vapor flow rate Q_{V} at every instant of the dispensing process.

This equality is obtained by means of a proportional solenoid valve 52 mounted on the vapor recovery line 51 upstream of the pump 50 and driven by an electronic control system 53 equipped with a microprocessor in order to regulate the flow rate Qᵥ.

This electronic control system 53 is connected to the encoder 41 or to the computer 42 in order to ensure that an instantaneous liquid flow rate Q_{L} is available at all times and to transmit an open command signal to the solenoid valve 52 which depends on this flow rate.

The command signal to be applied to the solenoid valve 52 depending on the liquid flow rate Q_{L} was determined beforehand during a phase of calibrating the installation and stored in memory in the microprocessor, in particular in the form of a table.

The recovery efficiency E % which is defined by the ratio 100(Qᵥ/Q_{L}) is never exactly equal to 100% in practice.

Consequently, the storage tank 20 is equipped with a vent 21 and is linked to the atmosphere by a two-way valve 22.

This system allows the vapor to escape if the pressure in the storage tank 20 is higher than a predetermined threshold, for example 20 mbar above atmospheric pressure P_{A}, or conversely allows air into the storage tank if the pressure within it is below a predetermined threshold and is, for example, 10 mbar below atmospheric pressure.

It should be pointed out that an installation of this type is capable of dispensing different types of fuel, in which case several dispensing guns 10 are provided, all of which are linked to the same solenoid valve 52.

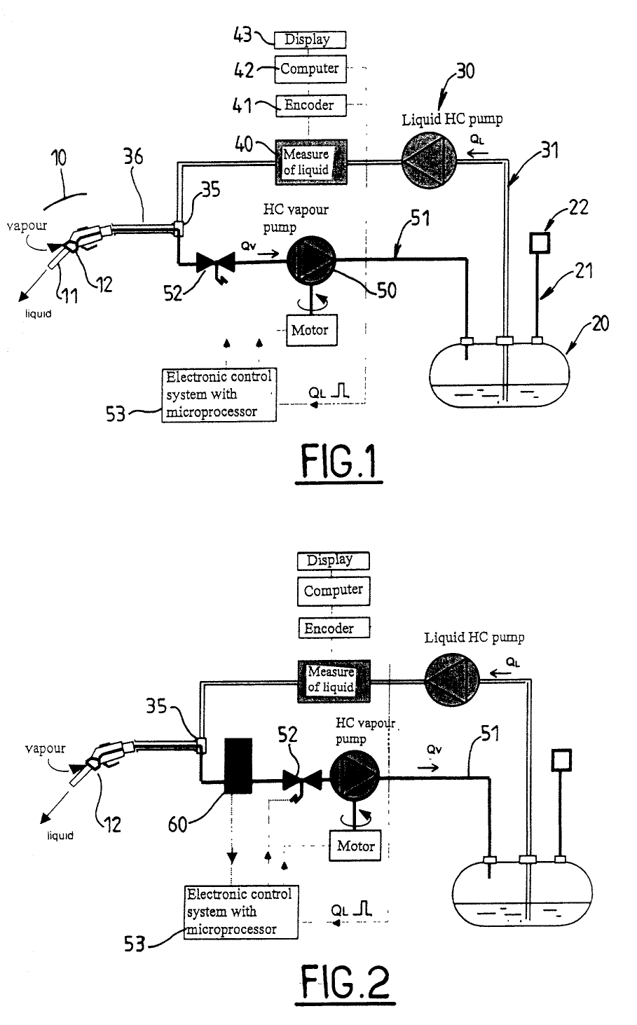

An example of a fuel dispensing installation such as proposed by the invention, equipped with a device for determining the hydrocarbon content of aspirated vapor, comprising a density flow meter on the one hand working in co-operation with a sensor for measuring relative pressure on the other, is illustrated in FIG. 2.

In this drawing, the device 60 for determining the hydrocarbon content of aspirated vapor is connected into the vapor recovery line 51 between the liquid/vapor separator 35 and the proportional solenoid valve 52.

The electronic control system 53 is linked to the device 60 and will therefore be supplied with instantaneous values for the vapor flow rate Q_{VLU} indicated by the flow meter on the one hand and the relative pressure δP supplied by the relative pressure sensor on the other.

For the purposes of the invention, the pressure sensor is generally of a construction which operates by reference to atmospheric pressure P_{A}; it therefore supplies information relating to δP which corresponds to the difference between the absolute pressure at the measurement point and atmospheric pressure.

In the installation illustrated in FIG. 2, because the vapor on a level with the annular orifice 12 of the dispensing gun 10 is sucked in at atmospheric pressure P_{A}, δP represents the drop in pressure in the part of the vapor recovery circuit disposed between the intake point, i.e. the dispensing gun 10, on the one hand and the device 60 on the other.

Clearly δP will be negative during suction, in effect:

δP−P*P_{A} and P<P_{A}

P_{A}: absolute atmospheric pressure

P: absolute pressure measured at the inlet of the flow meter.

It should be pointed out that the dispensing guns of conventional fuel dispensing installations are as a rule fitted with a valve connected into the vapor recovery circuit which does not open unless fuel is being dispensed.

The presence of this valve means that the installation cannot be recalibrated with air once it has been commissioned into service, after being initially calibrated with air.

However, in order to enable a subsequent automatic calibration, the invention offers an advantage whereby the installation may be fitted with two three-way solenoid valves actuated by the electronic control system.

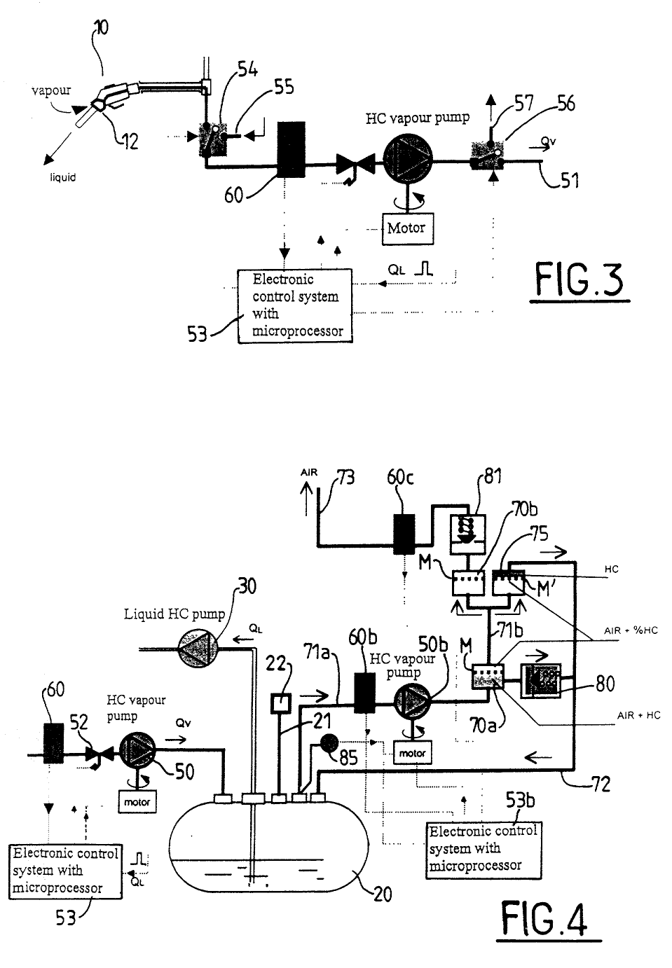

An example of an installation with this feature is illustrated in FIG. 3, which corresponds to a partial view of FIG. 2.

In this drawing, the vapor recovery line 51 is fitted with two three-way solenoid valves 54, 56, actuated by the electronic control system 53.

The first solenoid valve 54 enables either vapor to be sucked in through the annular orifice 12 of the dispensing gun 10 or air via its inlet 55.

The second solenoid valve 56 enables the aspirated vapor or air to be directed either to the storage tank 20 or to the atmosphere via its outlet 57.

During normal operation, when fueling, the electronic control system 53 actuates the solenoid valves 54 and 56 so that the aspirated vapor is conveyed to the storage tank 20.

The electronic control system 53 does not allow air to pass between the inlet 55 of the solenoid valve 54 and the outlet 57 of the solenoid valve 56 except during automatic calibration periods, i.e. outside of dispensing times.

The periodic automatic calibration operations run on such an installation in accordance with the first and second embodiments of the invention will be described below.

In accordance with the first embodiment of the invention, during the step of initially calibrating the installation with air, once the air resistance value R0 in the part of the vapor recovery circuit disposed between the dispensing gun 10 on the one hand and the device 60 for determining the hydrocarbon content of the aspirated vapor, i.e. the pressure sensor and the flow meter, on the other, has been determined, air is circulated between the inlet 55 of the first solenoid valve 54 and the outlet 57 of the second solenoid valve 56.

In a similar manner, the air resistance r0 is determined in the part of the vapor recovery circuit between the first solenoid valve 54 on the one hand and the device 60 for determining the hydrocarbon content of the aspirated vapor on the other.

This value r0 is also stored in memory.

During a periodic automatic calibration run, the electronic control system 53 issues a command to switch the solenoid valves 54 and 56 so that air is circulated between the inlet 55 of the first solenoid valve 54 and the outlet 57 of the second solenoid valve 56.

A new air resistance value r′0 is then determined, still in the same manner, for the part of the vapor recovery circuit between the first solenoid valve 54 and the device 60 for determining the hydrocarbon content of the aspirated vapor.

Using the value r′0 as a basis, a re-updated value R′0 is calculated for the air resistance in the part of the vapor recovery circuit between the dispensing gun 10 and the device 60 for determining the hydrocarbon content of the aspirated vapor, using the formula:

$${R}_{0}^{′} = {R}_{0} * \frac{{r}_{0}^{′}}{{r}_{0}}$$

After this automatic calibration, when fueling during normal operation, the same operations are reiterated in order to calculate the value of the vapor resistance R1 in the part of the vapor recovery circuit between the dispensing gun 10 and the device 60 for determining the hydrocarbon content of the aspirated vapor and a command or alarm is triggered or the installation is shut down if it is found that:

R1≦kR0 or R1≦k*r′0/r0*R0

Similarly, in accordance with the second embodiment of the invention, during the step of initially calibrating the installation with air, once the table T0[δP,Qᵥ] representing a characteristic linked to the drop in air pressure in the part of the vapor recovery circuit between the dispensing gun 10 and the device 60 for determining the hydrocarbon content of the aspirated vapor has been determined, air is circulated between the inlet 55 of the first solenoid valve 54 and the outlet 57 of the second solenoid valve 56.

A second table t0[δₚ,q_{V}] is then established in a similar manner representing this same characteristic linked to the drop in air pressure in the part of the vapor recovery circuit between the first solenoid valve 54 and the device 60 for determining the hydrocarbon content of the aspirated vapor and this second table is also stored in memory.

During the initial automatic calibration, the electronic control system 53 issues a command to switch the solenoid valves 54 and 56 so that air is circulated between the inlet 55 of the first solenoid valve 54 and the outlet 57 of the second solenoid valve 56.

The values for the air flow rate q′_{V} and the relative pressure δₚ are then measured and a search is run in the table t0[δₚ,qᵥ] to find the flow rate qᵥ such that q_{V}=q′_{V} in order to determine a ratio:

α=δₚ′/δₚ

The table T0[δP,Qᵥ] is then updated by multiplying all the pressure values by the coefficient a in order to obtain a new table T1[αP,Qᵥ].

Then, whilst fueling during normal operation, the same operations are reiterated, i.e. the values for the vapor flow rate Q_{VLU} and the relative pressure δP are measured at regular intervals, the real vapor flow rate Q_{V} is calculated on the basis of the vapor flow rate Q_{VLU}, after which, for each vapor flow rate Qᵥ, the table T1[αδP,Qᵥ] is searched to find the relative pressure αδPₐᵢᵣ corresponding to the same air flow rate.

The relative pressure values δP and αδPₐᵢᵣ, are then compared by calculating the factor λ defined by the equation:

$$λ = \frac{δ P - α δ {P}_{air}}{α δ {P}_{air}}$$

and a command or an alarm is triggered or the installation is shut down if it is found that:

λ≦λₑₓₚ≈0.063

The invention offers another feature whereby the temperature is corrected.

It should be pointed out that the temperature acts on the density ρ and on the viscosity μ of the aspirated vapor.

Accordingly, if, during dispensing, the ambient temperature is very different from that which prevailed during calibration, it is necessary to correct the reference parameters for the air in order to obtain more accurate air resistance values for R and the ratio λ.

The automatic calibration operation enables these parameters to be updated. Consequently, frequent automatic calibration can eliminate variations in ambient temperature.

However, for the purposes of the invention, the ambient temperature may be measured and corrections applied accordingly.

Another preferred feature of the invention resides in the fact of monitoring the hydrocarbon content of a vapor circulating in a system for purging the fuel storage tank of a fuel dispensing installation equipped with a system for recovering emitted vapor.

For the purposes of the invention, a purging system of this type comprises:

a vent linked to the atmosphere by a two-way valve system allowing vapor to escape if the pressure in the storage tank is above a predetermined threshold and allowing air to penetrate the storage tank if the pressure within the latter is below a predetermined threshold,

a vapor intake circuit comprising a suction pump enabling the vapor above the fuel in the storage tank to be circulated between the latter and the atmosphere at a vapor flow rate Q_{V},

an electronic control system equipped with a microprocessor co-operating with means for regulating the vapor flow rate Q_{V} and

elements for selectively filtering the air to ensure that the vapor discharged to the atmosphere via the vapor intake circuit is essentially free of hydrocarbons.

The purpose of an installation of this type is to eliminate the risk of localized pollution on a level with the vent of the storage tank when the pressure P_{C} in the latter becomes higher than atmospheric pressure P_{A}.

The method proposed by the invention enables monitoring to ensure that this installation is operating smoothly.

To this end, by virtue of another feature of the invention, a device for detecting the hydrocarbon content of the aspirated vapor is connected downstream of the selective air-filtering elements and a command or an alarm is triggered or the installation is shut down if the hydrocarbon content of the vapor discharged to the atmosphere by the vapor intake circuit is found to be higher than a predetermined threshold.

The method proposed by the invention also enables a check to be run to ensure that the hydrocarbon content of the storage tank above the fuel remains at a sufficient level to avoid reaching the limit of explosiveness.

In practice, this limit of explosiveness could conceivably be reached if the vapor recovery circuit were not fitted with a device for determining the hydrocarbon content of the aspirated hydrocarbons directly downstream of the dispensing gun.

To this end and by virtue of another feature of the invention, a device for detecting the hydrocarbon content of the aspirated vapor is connected upstream of the selective air-filtering elements and a command or an alarm is triggered or the installation is shut down if the hydrocarbon content of the aspirated vapor corresponding to the hydrocarbon content of the vapor above the fuel in the storage tank is found to be within a range which presents a risk of explosion.

Clearly, in either of the two situations described above, the hydrocarbon content of the aspirated vapor may be calculated on the basis of the two embodiments of the method proposed by the invention as described above.

As a result of another feature of the invention, the installation is fitted with a pressure controller or a pressure sensor sensitive to the vapor pressure prevailing in the storage tank in order to trigger an alarm if this pressure is located outside a predetermined range, which co-operates with the suction pump in order to issue a command to stop or start this pump if this pressure reaches predetermined threshold values.

By way of example, this pressure controller or this pressure sensor may enable:

a first alarm to be triggered if P_{C}≧δP_{A},

a second alarm to be triggered if P_{C}≦P_{A}−c1,

c1 being a first reference value which in particular is equal to approximately 10 mb indicating that air is starting to get into the tank through the two-way valve,

a command to be issued to stop the suction pump if P_{C}≦P_{A}−c²,

c2 being a second reference value, in particular approximately 8 mb,

a command to be issued to re-start the suction pump if P_{C}≧P_{A}−c^{3,}

c3 being a third reference value, in particular in the order of 2 mb.

Another feature of the invention is that the installation is fitted with a pressure sensor sensitive to the vapor pressure P_{C} prevailing in the storage tank and co-operating with the electronic control system in order to apply a correction to the detected value of the hydrocarbon content of the vapor discharged to the atmosphere by the vapor intake circuit and/or the vapor above the fuel in the storage tank depending on the difference between the pressure P_{C} prevailing in the storage tank and atmospheric pressure P_{A}.

The purpose of this correction is to take account of the fact that the vapor intake circuit takes in vapor not at atmospheric pressure P_{A} but at the pressure P_{C} of the storage tank.

The sensor therefore supplies data relating to the relative pressure Pᵢₙ=P_{C}−P_{A}.

In the case of the first embodiment of the invention, the resistance R by reference to atmospheric pressure was written:

$$R = \frac{δ P}{{Q}_{V}^{x}} = \frac{P1 - {P}_{A}}{{Q}_{V}^{x}}$$

When the aforementioned correction is taken into account, the resistance value becomes:

$$R = \frac{P1 - {P}_{c}}{{Q}_{V}^{x}} = \frac{[ ( P1 - {P}_{A} ) - ( {P}_{c} - {P}_{A} ) ]}{{Q}_{V}^{x}} = \frac{δ P - {P}_{in}}{{Q}_{V}^{x}}$$

Similarly, with the second embodiment of the invention, the parameter λ after correction is defined by the equation:

$$λ = \frac{( δ P - {P}_{in} ) vap - {( δ P - {P}_{in} )}_{air}}{{( δ P - {P}_{in} )}_{air}}$$

As a result of another feature of the invention, the selective air filtering elements incorporate two stages of filtration.

The first filtration stage comprises a first selective air filter co-operating with a valve calibrated so as to transfer the air-enriched vapor flow to the second filtration stage and a part of the flow enriched with hydrocarbons to the storage tank.

The second filtration stage in turn comprises firstly a second selective air filter, which is preferably identical to the first selective air filter, co-operating with a check valve so that the air-enriched vapor flow is transferred to the atmosphere and secondly a selective hydrocarbon filter enabling the flow enriched with hydrocarbons to be returned to the storage tank.

An example of an installation with these fixtures is illustrated in FIG. 4 which shows part of FIGS. 2 and 3.

In this drawing, the storage tank 20 is provided with a vent 21 and is connected to the atmosphere via a two-way valve system 22.

This installation is fitted with a vapor intake circuit comprising a suction pump 50b enabling the vapor above the fuel in the storage tank 20 to be circulated between the latter and the atmosphere at a vapor flow rate Q_{V}.

The suction pump 50b may be a fixed speed pump but is preferably a variable speed pump driven by an electronic control system 53b provided with a microprocessor so that the flow rate Q_{V} can be varied and can be so in order to adjust to the requirements of the installation—it also being possible to obtain a variable flow rate by using a proportional valve such as 52.

The pump 50b sucks the vapor into the tank 20 via a line 71a into which a device 60b is connected for determining the hydrocarbon content of the aspirated vapor, comprising the combination of a flow meter and a sensor for measuring relative pressure.

This pump 50b supplies selective air filtering elements incorporating two filtration stages.

The first filtration stage comprises a first selective air filter 70a, the membrane M of which essentially allows air to pass through (99% and 1% hydrocarbons, for example).

The air-enriched flow is directed to the second filtration stage by a line 71b.

A part of the flow enriched with hydrocarbons is returned to the storage tank 20 by a line 72 fitted with a calibrated valve 80.

This valve 80 maintains an above-atmospheric pressure below the membrane M of the filter 70a to promote the transfer of the filtered flow to line 71b.

Outside its calibrated pressure, the valve 80 opens and allows some of the flow enriched with hydrocarbons to pass through to line 72.

The second filtration stage consists of two filters connected in parallel, namely a second selective air filter 70b identical to the first filter 70a on the one hand and a filter 75 which allows only hydrocarbons to pass through on the other.

At the outlet of the second filter 70b, the proportion of air in the flow escaping to the atmosphere is in the order of 99.99%.

This air is discharged via a line 73 to which a check valve 81 is connected as well as a device 60c for determining the hydrocarbon content of aspirated vapor, which also consists of a flow meter combined with a pressure sensor.

The selective hydrocarbon filter 75 is fitted with a selective membrane M′ which allows only hydrocarbons to pass through, which can then be returned to the storage tank 20 via line 72.

As illustrated in FIG. 4, this installation is also fitted with a pressure controller or a pressure sensor 85 sensitive to the vapor pressure prevailing in the storage tank 20.

Although not illustrated in this drawing, the installation may also be fitted with two sets of solenoid valves to enable the periodic automatic calibration thereof.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method of controlling the hydrocarbon content of a mixture of air/hydrocarbon vapor circulating from an intake point into a fuel dispensing installation equipped with a vapor recovery or suction system, the method comprising the steps of:
connecting a device to a vapor intake circuit for determining the hydrocarbon content of the aspirated vapor comprising a combination of a flow meter on the one hand and a sensor for measuring relative pressure by reference to atmospheric pressure P_{A} on the other;
connecting said device to an electronic control system to enable the electronic control system to generate instantaneous values for the vapor flow rate Q_{VLU} indicated by the flow meter on the one hand and the relative pressure δP indicated by the pressure sensor on the other, representing the loss in pressure in the part of the vapor intake circuit disposed between the intake point on the one hand and the pressure sensor and flow meter on the other;
calibrating the electronic control system with air beforehand in order to determine a characteristic linked to the loss in air pressure in the part of the vapor intake circuit disposed between the intake point on the one hand and the pressure sensor and flow meter on the other and storing this characteristic in memory;
measuring at regular intervals the values of the vapor flow rate Q_{VLU} and the relative pressure δP during normal operation;
calculating the real instantaneous flow rate by the formula:
$${Q}_{V} = {Q}_{VLU} * ( \frac{δ P}{{P}_{A}} + 1 )$$
determining the hydrocarbon content of the vapor circulating in the vapor intake circuit by use of the density ρ and the viscosity μ of the vapor, which are derived from the characteristic linked to the loss in air pressure stored in memory beforehand; and
issuing a command if the hydrocarbon content is found to be within a predetermined range.

2. A method of controlling the hydrocarbon content of a mixture of air/hydrocarbon vapor circulating from an intake point into a system for purging a fuel storage tank of a fuel dispensing installation equipped with a system for recovering emitted vapor, the method comprising the steps of:
providing a vent linked to the atmosphere by a system of directional valves allowing vapor to escape if the pressure in the storage tank is above a predetermined threshold and allowing air to penetrate the storage tank if the pressure within the latter is below a predetermined threshold;
providing a vapor intake circuit comprising a suction pump enabling the vapor above the fuel in the storage tank to be circulated between the latter and the atmosphere at a vapor flow rate Qᵥ;
providing an electronic control system equipped with a microprocessor co-operating with means for regulating the vapor flow rate Q_{V};
providing elements for selectively filtering the air to ensure that the vapor discharged to the atmosphere via the vapor intake circuit is essentially free of hydrocarbons;
connecting a device to the vapor intake circuit of the purging system for determining the hydrocarbon content of the aspirated vapor comprising a combination of a flow meter on the one hand and a pressure sensor for measuring relative pressure by reference to atmospheric pressure P_{A} on the other;
connecting said device to the electronic control system to enable it to generate instantaneous values for the vapor rate Q_{VLU} indicated by the flow meter on the one hand and the relative pressure δP indicated by the pressure sensor on the other, representing the loss in pressure in the part of the vapor intake circuit disposed between the intake point on the one hand and the pressure sensor and flow meter on the other;
calibrating said device with air beforehand in order to determine a characteristic linked to the loss in air pressure in the part of the vapor intake circuit disposed between the intake point on the one hand and the pressure sensor and flow meter on the other and storing a characteristic in memory;
measuring at regular intervals the values of the vapor flow rate Q_{VLU} and the relative pressure δP;
calculating the real instantaneous flow rate by the formula:
$${Q}_{V} = {Q}_{VLU} * ( \frac{δ P}{{P}_{A}} + 1 )$$
determining the hydrocarbon content of the vapor circulating in the vapor intake circuit by use of the density ρ and the viscosity μ of the vapor, which are derived from the characteristic linked to the loss in air pressure stored in memory beforehand; and
issuing a command if the hydrocarbon content is found to be within a predetermined range.

3. A method as claimed in claim 1, in that the characteristic linked to the drop in air pressure in the part of the vapor intake circuit disposed between the intake point on the one hand and the pressure sensor and flow meter on the other is the resistance R defined by the equation:
$$R = \frac{δ P}{{Q}_{V}^{x}}$$
in which
δP represents the loss in pressure expressed in Pascal,
Q_{V} represents the vapor flow rate expressed in m³/s and
x represents a parameter equal to 7/4 in theory and approximately 1.8 in practice,
the drop in pressure δP being further defined by the equation:
$$δ P = C [ \frac{L * {ρ}^{3 / 4} * {Q}_{V}^{x} * {μ}^{1 / 4}}{{d}^{19 / 4}} ]$$
in which:
L represents the length of the part of the circuit in question expressed in meters,
d represents the diameter in question, being a constant of this part of the circuit, expressed in meters,
μ represents the viscosity of the vapor expressed in Pa.s,
ρ represents the density of the vapor expressed in g/l and
C represents a parameter equal to 0.2414.

4. A method as claimed in claim 3, including the following sequence of steps:
computing a table T[Qᵥ/Q_{V}^{x}] in which a value Q_{V}^{x} is correlated with different vapor flow rates Q_{V} between 0 and Q_{VMAX} and this table is stored in memory;
during the prior step of calibrating the installation with air, the suction pump is activated and the regulating means are controlled in order to obtain several different vapor flow rates Qᵥ;
measuring the relative pressure δP corresponding to these vapor flow rates Q_{V}, and a value for the air resistance R in the part of the vapor intake circuit disposed between the intake point on the one hand and the pressure sensor and flow meter on the other is derived from the table T[Qᵥ,Q_{V}^{x}];
calculating the average R0 of the different values R thus obtained and storing in memory,
measuring at regular intervals during normal operation, the values of the vapor flow rate Q_{VLU} and the relative pressure δP;
calculating the real vapor flow rate Q_{V} from the vapor flow rate Q_{VLU} using the formula:
$${Q}_{V} = {Q}_{VLU} * ( \frac{δ P}{{P}_{A}} + 1 )$$
where the value Q_{V}^{x} is derived from the table T[Qᵥ,Q_{V}^{x}], the value of the vapor resistance R1 in the part of the vapor intake circuit disposed between the intake point on the one hand and the pressure sensor and flow meter on the other is calculated, the vapor resistance R1 is compared with the air resistance R0; and
a command or an alarm is triggered or the installation is shut down if the ratio R1/R0 is found to be within a predetermined range, in particular if it is found that:
R1≦kR0
the parameter k being a parameter which allows the upper limit of explosiveness, corresponding to a vapor Vₑₓₚ with an 8% hydrocarbon content, to be taken into account and being defined by the equation:
$$k = {( \frac{{P}_{V exp}}{{P}_{air}} )}^{3.4} {( \frac{{μ}_{V exp}}{{μ}_{air}} )}^{1 / 4} \approx 1.063 .$$

5. A method as claimed in claim 1, including the following sequence of steps:
during the prior step of calibrating the installation with air, the suction pump is activated and the regulating means are activated step by step so as to vary the air flow circulating in the vapor intake circuit;
with each step, the values of the vapor flow rate Q_{VLU} and the relative pressure δP are measured;
the real vapor flow rate Q_{V} is calculated from the vapor flow rate Q_{VLU} using the formula:
$${Q}_{V} = {Q}_{VLU} ( \frac{δ P}{{P}_{a}} + 1 )$$
a table T0[δP,Qᵥ] is established, representing the characteristic linked to the drop in air pressure in the part of the vapor intake circuit disposed between the intake point on the one hand and the pressure sensor and flow meter on the other and this table T0[δP,Qᵥ] is stored in memory;
during normal operation, the values of the vapor flow rate Q_{VLU} and relative pressure δP are measured at regular intervals;
the real vapor flow rate Q_{V} is calculated from the vapor flow rate Q_{VLU} by the formula:
$${Q}_{V} = {Q}_{VLU} ( \frac{δ P}{{P}_{a}} + 1 )$$
for each vapor flow rate Qᵥ, the table T0[δP,Qᵥ] is searched for a relative pressure δPₐᵢᵣ corresponding to the same rate of air flow;
the relative pressures δP and δPₐᵢᵣ are compared by calculating a factor λ defined by the equation:
$$λ = \frac{δ P - δ {P}_{air}}{δ {P}_{air}}$$
the relative pressure δP, corresponding to the drop in pressure in the part of the vapor intake circuit disposed between the intake point on the one hand and the pressure sensor and flow meter on the other, also being defined by the equation:
$$δ P = C [ \frac{L * {ρ}^{3 / 4} * {Q}_{V}^{x} * {μ}^{1 / 4}}{{d}^{19 / 4}} ]$$
in which, if δP is expressed in Pascals,
L represents the length of the part of the circuit in question expressed in m,
d represents the diameter in question, being a constant of this part of the circuit, expressed in m,
μ represents the viscosity of the vapor expressed in Pa.s,
ρ represents the density of the vapor expressed in g/l,
C represents a parameter equal to 0.2414,
Q_{V} represents the vapor flow rate expressed in m³/s and
x represents a parameter equal to 7/4 in theory and approximately 1.8 in practice,
the factor λ then also being defined by the equation:
$$λ = \frac{{( {ρ}^{3 / 4} * {μ}^{1 / 4} )}_{vapor}}{{( {ρ}^{3 / 4} * {μ}^{1 / 4} )}_{air}} - 1 ; and$$
a command or alarm is triggered if λ is found to be within a predetermined range, in particular if it is found that:
λ≦λₑₓₚ≈0.063
λₑₓₚ being the value of λ corresponding to a vapor Vₑₓₚ with an 8% hydrocarbon content corresponding to the upper limit of explosiveness.

6. A method as claimed in claim 1 in which a periodic automatic calibration of the installation is run with air in order to update the characteristic linked to the loss in air pressure in the part of the vapor intake circuit disposed between the intake point on the one hand and the pressure sensor and flow meter on the other.

7. A method as claimed in claim 1 in which the effects of the vapor temperature are corrected for.

8. A method as claimed in claim 6 in which automatic calibrations with air are repeated at a sufficient frequency to correct the temperature and the associated sensor readings.

9. A method as claimed in claim 2 in which a device for detecting the hydrocarbon content of the aspirated vapor is connected downstream of selective air filtering elements and a command or an alarm is triggered or the installation is shut down if the hydrocarbon content of the vapor discharged to the atmosphere by the vapor intake circuit is found to be above a predetermined threshold.

10. A method as claimed in claim 2 in which a device for detecting the hydrocarbon content of the aspirated vapor is connected upstream of selective air filtering elements and a command or an alarm is triggered or the installation is shut down if the hydrocarbon content of the aspirated vapor corresponding to the hydrocarbon content of vapor above the fuel in the storage tank is within a range presenting a risk of explosion.

11. A method as claimed in claim 2 in which the installation is fitted with a pressure controller or a pressure sensor sensitive to the pressure prevailing in the storage tank in order to trigger an alarm if this pressure is outside a predetermined range, which co-operates with the suction pump or purging system to issue a command to stop or start this pump if this pressure reaches predetermined threshold values.

12. A method as claimed in claim 2 in that the installation is fitted with a pressure sensor sensitive to the pressure prevailing in the storage tank and co-operating with the electronic control system to correct the factor λ or the resistance R and hence the detected value of the hydrocarbon content discharged to the atmosphere by the vapor intake circuit and/or the vapor above the fuel in the storage tank depending on the difference between the pressure prevailing in the storage tank and atmospheric pressure.

13. A method as claimed in claim 2 which the selective air filtering elements incorporate two filtration stages, the first filtration stage comprising a first selective air filter co-operating with a calibrated valve so as to transfer the air-enriched vapor flow to the second filtration stage and return some of the flow enriched with hydrocarbons to the storage tank, the second filtration stage comprising a second selective air filter, preferably identical to the first selective air filter, co-operating with a check valve in order to transfer the air-enriched vapor flow to the atmosphere on the one hand and a selective hydrocarbon filter enabling the flow enriched with hydrocarbons to be returned to the storage tank, on the other.

14. A method as claimed in claim 2, in that the characteristic linked to the drop in air pressure in the part of the vapor intake circuit disposed between the intake point on the one hand and the pressure sensor and flow meter on the other is the resistance R defined by the equation:
$$R = \frac{δ P}{{Q}_{V}^{x}}$$
in which
δP represents the loss in pressure expressed in Pascal,
Q_{V} represents the vapor flow rate expressed in m³/s and
x represents a parameter equal to 7/4 in theory and approximately 1.8 in practice,
the drop in pressure δP being further defined by the equation:
$$δ P = C [ \frac{L * {ρ}^{3 / 4} * {Q}_{V}^{x} * {μ}^{1 / 4}}{{d}^{19 / 4}} ]$$
in which:
L represents the length of the part of the circuit in question expressed in meters,
d represents the diameter in question, being a constant of this part of the circuit, expressed in meters,
μ represents the viscosity of the vapor expressed in Pa.s,
ρ represents the density of the vapor expressed in g/l and
C represents a parameter equal to 0.2414.

15. A method as claimed in claim 2, including the following sequence of steps:
during the prior step of calibrating the installation with air, the suction pump is activated and the regulating means are activated step by step so as to vary the air flow circulating in the vapor intake circuit;
with each step, the values of the vapor flow rate Q_{VLU} and the relative pressure δP are measured;
the real vapor flow rate Q_{V} is calculated from the vapor flow rate Q_{VLU} using the formula:
$${Q}_{V} = {Q}_{VLU} ( \frac{δ P}{{P}_{a}} + 1 )$$
a table T0[δP,Qᵥ] is established, representing the characteristic linked to the drop in air pressure in the part of the vapor intake circuit disposed between the intake point on the one hand and the pressure sensor and flow meter on the other and this table T0[δP,Qᵥ] is stored in memory;
during normal operation, the values of the vapor flow rate Q_{VLU} and relative pressure δP are measured at regular intervals;
the real vapor flow rate Q_{V} is calculated from the vapor flow rate Q_{VLU} by the formula:
$${Q}_{V} = {Q}_{VLU} ( \frac{δ P}{{P}_{a}} + 1 )$$
for each vapor flow rate Qᵥ, the table T0[δP,Qᵥ] is searched for a relative pressure δPₐᵢᵣ corresponding to the same rate of air flow;
the relative pressures δP and δPₐᵢᵣ are compared by calculating a factor λ defined by the equation:
$$λ = \frac{δ P - δ {P}_{air}}{δ {P}_{air}}$$
the relative pressure δP, corresponding to the drop in pressure in the part of the vapor intake circuit disposed between the intake point on the one hand and the pressure sensor and flow meter on the other, also being defined by the equation:
$$δ P = C [ \frac{L * {ρ}^{3 / 4} * {Q}_{V}^{x} * {μ}^{1 / 4}}{{d}^{19 / 4}} ]$$
in which, if δP is expressed in Pascals,
L represents the length of the part of the circuit in question expressed in m,
d represents the diameter in question, being a constant of this part of the circuit, expressed in m,
μ represents the viscosity of the vapor expressed in Pa.s,
ρ represents the density of the vapor expressed in g/l,
C represents a parameter equal to 0.2414,
Q_{V} represents the vapor flow rate expressed in m³/s and
x represents a parameter equal to 7/4 in theory and approximately 1.8 in practice,
the factor λ then also being defined by the equation:
$$λ = \frac{{( {ρ}^{3 / 4} * {μ}^{1 / 4} )}_{vapor}}{{( {ρ}^{3 / 4} * {μ}^{1 / 4} )}_{air}} - 1 ; and$$
a command or alarm is triggered if λ is found to be within a predetermined range, in particular if it is found that:
λ≦λₑₓₚ≈0.063
λₑₓₚ being the value of λ corresponding to a vapor Vₑₓₚ with an 8% hydrocarbon content corresponding to the upper limit of explosiveness.

16. A method as claimed in claim 2 in which a periodic automatic calibration of the installation is run with air in order to update the characteristic linked to the loss in air pressure in the part of the vapor intake circuit disposed between the intake point on the one hand and the pressure sensor and flow meter on the other.
